(19) United States
(12) Patent Application Publication
(10) Pub. No.: US 2024/0071580 A1
(43) Pub. Date: Feb. 29, 2024

(54) MEDICAL INFORMATION PROCESSING APPARATUS, MEDICAL INFORMATION PROCESSING SYSTEM, MEDICAL INFORMATION PROCESSING METHOD, AND RECORDING MEDIUM

(71) Applicant: KONICA MINOLTA, INC., Tokyo (JP)

(72) Inventor: Hiroshi TERAO, Tokyo (JP)

(73) Assignee: KONICA MINOLTA, INC., Tokyo (JP)

(21) Appl. No.: 18/229,707

(22) Filed: Aug. 3, 2023

(30) Foreign Application Priority Data
Aug. 23, 2022 (JP) 2022-132653

(51) Int. Cl. G16H 10/60; G16H 15/00; G16H 30/20

(52) U.S. Cl. CPC G16H 10/60; G16H 15/00; G16H 30/20

(57) ABSTRACT

A medical information processing apparatus includes a hardware processor that sets first checking authority and second checking authority. The first checking authority is authority to check an interpretation report created by a first user who has interpreted a medical image of a subject. The second checking authority is authority to check the interpretation report and is different from the first checking authority.

### CROSS-REFERENCE TO RELATED APPLICATIONS

The entire disclosure of Japanese Patent Application No. 2022-132653, filed on Aug. 23, 2022, including description, claims, drawings and abstract is incorporated herein by reference.

### BACKGROUND

### Technical Field

The present invention relates to a medical information processing apparatus, a medical information processing system, a medical information processing method, and a recording medium.

### Description of Related Art

In recent years, computerization of information has progressed in the medical field, and medical information such as medical images and medical reports is managed as electronic data. In a case where a certain doctor (a requesting doctor) requests a radiologist to interpret a medical image and the radiologist creates an interpretation report, the requesting doctor checks the reading report and uses it for a diagnosis.

Conventionally, there is known an invention for managing information indicating that the interpretation report has been checked (read).

For example, the following medical information processing apparatus is described in Japanese Unexamined Patent Publication No. 2019-185418. Specifically, a checking operation indicating that the interpretation report has been checked is requested to a requesting doctor who requests the radiologist to create the interpretation report or a doctor belonging to a group to which the requesting doctor belongs. When the operation is performed, information indicating that reading has been performed is associated with the interpretation report.

From the viewpoint of team medical care, in a case where a doctor and a nurse form a team for treatment, there is a demand that not only the doctor but also the nurse wants to check an interpretation report. In a case where the requesting doctor and a doctor in another medical department form a team for treatment, there is a demand that not only the requesting doctor but also the doctor in the other medical department want to check the interpretation report.

### SUMMARY

However, in the invention disclosed in Japanese Unexamined Patent Publication No. 2019-185418, only the checking operation is requested to the requesting doctor or the doctor belonging to the same group as the requesting doctor. That is, with the invention described in Japanese Unexamined Patent Publication No. 2019-185418, it is not possible to give the authority to check an interpretation report to nurses, doctors in other medical departments, and the like.

An object of the present invention is to provide a medical information processing apparatus, a medical information processing system, a medical information processing method, and a recording medium capable of check of an interpretation report in accordance with needs.

To achieve at least one of the abovementioned objects, according to an aspect of the present invention, medical information processing apparatus reflecting one aspect of the present invention includes:
a hardware processor that sets first checking authority and second checking authority, the first checking authority being authority to check an interpretation report created by a first user who has interpreted a medical image of a subject, the second checking authority being authority to check the interpretation report and being different from the first checking authority.

To achieve at least one of the abovementioned objects, according to an aspect of the present invention, medical information processing system reflecting one aspect of the present invention includes:
a hardware processor that sets a first checking authority and a second checking authority, the first checking authority being authority to check an interpretation report created by a first user who has interpreted a medical image of a subject, the second checking authority being authority to check the interpretation report and being different from the first checking authority.

To achieve at least one of the abovementioned objects, according to an aspect of the present invention, medical information processing method reflecting one aspect of the present invention includes
setting a first checking authority and a second checking authority, the first checking authority being an authority to check an interpretation report created by a first user who has interpreted a medical image of a subject, the second checking authority being an authority to check the interpretation report and being different from the first checking authority.

To achieve at least one of the abovementioned objects, according to an aspect of the present invention, a non-transitory computer-readable recording medium stores a program that causes a hardware processor of a computer to set a first checking authority and a second checking authority, the first checking authority being an authority to check an interpretation report created by a first user who has interpreted a medical image of a subject, the second checking authority being an authority to check the interpretation report and being different from the first checking authority.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a system configuration diagram of a medical information processing system according to an embodiment of the present invention;

FIG. 2 is a block diagram showing a functional configuration of a medical information processing apparatus;

FIG. 3 is a diagram illustrating a data structure of a facility information table, a division information table, a user information table, and a user attribute table;

FIG. 4 is a diagram showing a data configuration of a patient information table, a report information table, a report check history table, and a report reference history table;

FIG. 5 is a block diagram illustrating a functional configuration of a client terminal;

FIG. 6 is a flowchart illustrating a report creating process executed in the medical information processing system:

FIG. 7 is an example of an interpretation flow editing screen;

FIG. 8 is an example illustrating a case of changing the setting of checking authority;

FIG. 9 is an example showing a case of changing a user having first checking authority;

FIG. 10 is an example showing a case of adding a user having second checking authority;

FIG. 11 is an example showing a case where a user having second checking authority is added;

FIG. 12 is a flowchart illustrating report checking status management processing:

FIG. 13 is an example of a report display screen;

FIG. 14 is an example of a report display screen;

FIG. 15 is an example of a report display screen; and

FIG. 16 is an example of a checking authority setting screen.

### DETAILED DESCRIPTION

Hereinafter, embodiments of the present invention will be described in detail with reference to the drawings. However, the scope of the invention is not limited to the disclosed embodiments.

<1. Configuration of Medical Information Processing System>

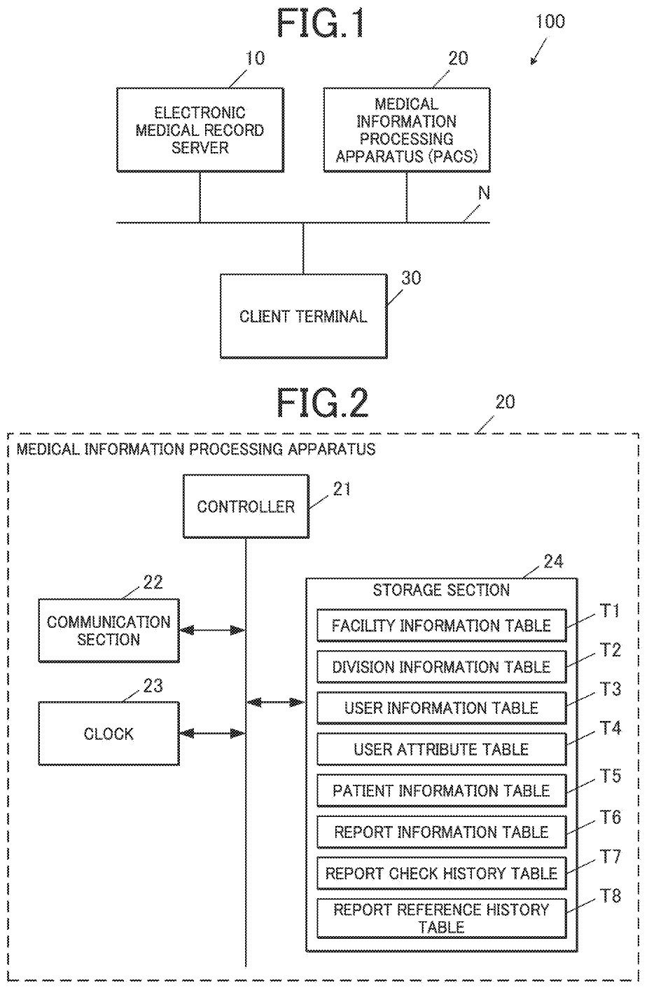

FIG. 1 illustrates an example of a system configuration of the medical information processing system 100.

As illustrated in FIG. 1, the medical information processing system 100 includes an electronic medical record server 10, a medical information processing apparatus 20, and a client terminal 30. The respective apparatuses included in the medical information processing system 100 are connected to each other via a communication network N such as a local area network (LAN) or a wide area network (WAN) so as to transmit and receive data.

Each apparatus included in the medical information processing system 100 conforms to the Health Level Seven (HL7) and Digital Image and Communications in Medicine (DICOM) standards. Communication between the apparatuses included in the medical information processing system 100 is performed in accordance with HL7 or DICOM.

The medical information processing system 100 includes a plurality of client terminals 30.

The electronic medical record server 10 generates electronic medical record information, order information, and the like in response to an operation instruction from the client terminal 30 used by a doctor. The electronic medical record information includes records of medical procedures, diagnosis results, and the like for a patient. The order information is information for requesting inspection of a patient, report creation for a medical image, and the like.

The electronic medical record system includes an electronic medical record server 10 and the client terminal 30.

The medical information processing apparatus 20 is a computer apparatus that manages medical information. The medical information processing apparatus 20 is, for example, a picture archiving and communication system (PACS).

The medical information is information related to medical care, and is information indicating findings, diagnosis contents, progress, and/or inspection results of a patient. The medical information includes, for example, a medical report and various kinds of information forming the medical report. The various kinds of information include patient information, inspection information, medical images (inspection images, measurement result images), measurement results, findings, and the like.

The medical report is a report in which information indicating findings, diagnosis contents, progress, and/or inspection results of a patient is described. A medical report may include, for example, patient information, inspection information, a medical image (an inspection image or a measurement result image), an enlarged/reduced image of the medical image, a measurement result, findings, and a header or footer. For each item of the medical report, an entry/paste area, arrangement thereof, and the like may be specified as a format.

The medical report is, for example, an interpretation report of medical image data, an inspection report, or the like.

The interpretation report is a report in which findings on a medical image captured by an imaging apparatus by a radiologist in a radiology division or the like are summarized in response to an inspection request from a doctor. The imaging device is any of various modalities such as CT and MRI, for example. The findings on the medical image are the presence or absence of abnormality, the condition of a patient, the indication of a disease, and the like. The interpretation report includes, for example, contents such as a type of lesion, an anatomical division, a degree, a situation, and a conclusion.

The inspection report is a report on the inspection of the patient. The inspection report is, for example, a specimen report regarding a specimen (blood or the like) collected from the patient, a bacteria report regarding bacteria in the patient, a physiological report regarding physiological information of the patient, or the like.

The patient information includes information on a patient. The patient information includes, for example, a patient name, a patient ID, a date of birth, and a sex.

The inspection information includes information on the inspection. The inspection information includes, for example, an inspection item, an inspection result (numerical value), a combination thereof, an inspection ID, an inspection date and time, an inspection description, an inspection instance UID, and the like.

A medical image is an image obtained by imaging a result of imaging or measuring a human body for medical care or medicine, and is an image used for diagnosis. The medical image is, for example, an X-ray image, an MRI image, a CT image, a pathological image, or the like, and may be a schema (a handwritten image) having information equivalent to those.

The measurement result is a result obtained by performing various medical measurements from the inspection information, the medical image, and the like. The measurement result includes, for example, a result of cardiothoracic ratio measurement, a result of measurement of visceral fat areas, and the like.

The findings include a judgment, an opinion, and the like regarding a result of asking a question to a patient and a result of examining a body and/or a medical image in order for a doctor to judge a medical condition of the patient. The findings include, for example, a physical finding. The findings of the medical report include judgment, opinion, and the like related to the result of examining the medical image.

The header and the footer include, for example, a logo, a name, a location, a telephone number, and email address of the hospital.

<1-1. Configuration of Medical Information Processing Apparatus>

FIG. 2 shows a functional configuration of the medical information processing apparatus 20.

As illustrated in FIG. 2, the medical information processing apparatus 20 includes a controller 21, a communication section 22, a clock 23, and a storage section 24. Each unit of the medical information processing apparatus 20 is connected by a bus.

The controller 21 includes a central processing unit (CPU), a read only memory (ROM), a random access memory (RAM), and the like. The controller 21 comprehensively controls processing operation of each section of the medical information processing apparatus 20. Specifically, the CPU reads various processing programs stored in the ROM, develops the programs in the RAM, and performs various processes in cooperation with the programs.

The communication section 22 includes a network interface and the like. The communication section 22 transmits and receives data to and from an external device connected via the communication network N.

The clock 23 includes a clocking circuit (RTC: Real Time Clock), clocks the current date and time by the clocking circuit, and outputs the current date and time to the controller 21.

The storage section 24 includes a hard disk drive (HDD), a nonvolatile semiconductor memory, or the like, and stores various types of data. For example, the storage section 24 stores medical information such as a medical report and a medical image generated by inspection of a patient.

The storage section 24 stores a facility information table T1, a division information table T2, a user information table T3, and a user attribute table T4.

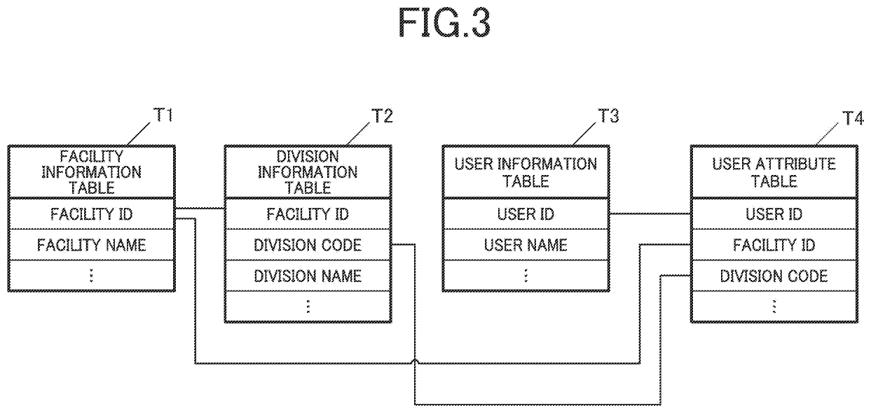

FIG. 3 illustrates data structures of the facility information table T1, the division information table T2, the user information table T3, and the user attribute table T4.

The facility information table T1 stores information related to the medical facility. In the facility information table T1, a facility ID, a facility name, and the like are associated with each medical facility. The facility ID is identification information given to the medical facility. The facility name is a name of the medical facility.

The division information table T2 stores information on the divisions (medical department) classified in the medical facility. In the division information table T2, a facility ID, a division code, a division name, and the like are associated with each division of each medical facility. The facility ID is a facility ID of a medical facility to which the division belongs. The division code is identification information given to a division. The division name is a name of a division.

The user information table T3 stores information on users who use the medical information processing apparatus 20. The user is a doctor, a nurse, or the like. In the user information table T3, a user ID, a user name, and the like are associated with each user.

The user ID is identification information assigned to the user.

The user name is a display name used when a user uses the medical information processing apparatus 20.

The user attribute table T4 stores attribute information (affiliation, position, etc.) of a user. In the user attribute table T4, a user ID, a facility ID, a division code, and the like are associated with each user. The facility ID is a facility ID of a medical facility to which the user belongs. The division code is a division code of a division to which the user belongs.

Since a single doctor may belong to a plurality of divisions, a plurality of kinds of attribute information can be registered for a single user in the user attribute table T4.

The controller 21 acquires staff information (user information, user attributes, and the like) of each medical facility from the electronic medical record server 10 via the communication section 22. Next, the controller 21 registers the acquired staff information in the user information table T3 and the user attribute table T4.

The storage section 24 further stores a patient information table T5, a report information table T6, a report check history table T7, and a report reference history table T8.

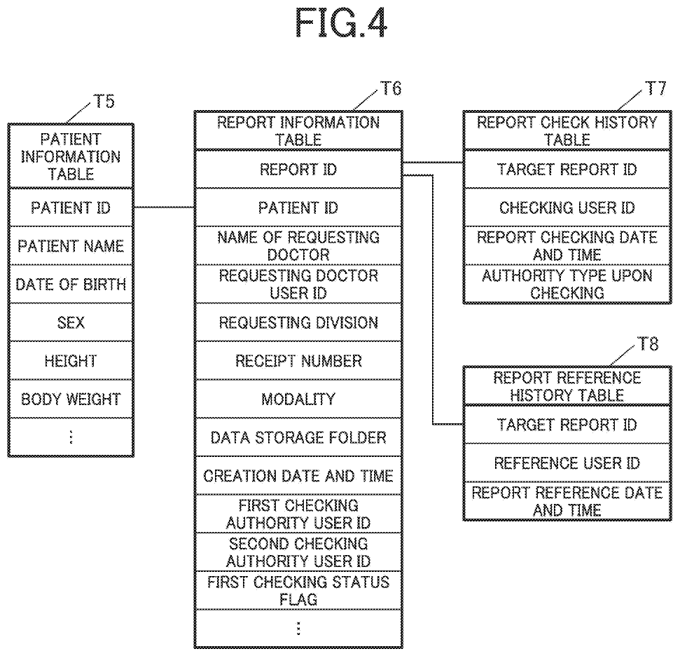

FIG. 4 illustrates data structures of the patient information table T5, the report information table T6, the report check history table T7, and the report reference history table T8.

The patient information table T5 stores information on a patient. In the patient information table T5, a patient ID, a patient name, a date of birth, a sex, a height, a body weight, and the like are associated with each patient. The patient ID is identification information given to a patient. The patient name, date of birth, sex, height, and body weight are the name, date of birth, sex, height, and body weight of the patient, respectively.

The report information table T6 stores information related to medical reports. In the report information table T6, for each medical report, a report ID, a patient ID, a requesting doctor name, a requesting doctor user ID, a requesting division, a receipt number, a modality, a data storage folder, a creation date and time, a first checking authority user ID, a second checking authority user ID, a first checking status flag, and the like are associated. The report ID is identification information given to the medical report. The patient ID is the patient ID of the patient related to the medical report. The name of requesting doctor is the name of the doctor (requesting doctor) who has requested to create the medical report. The requesting doctor user ID is a user ID of a doctor who has requested to create the medical report. The requesting division is the division (medical department) that has requested to create a medical report. Usually, the requesting division is a division to which the requesting doctor belongs. The receipt number is a receipt number given to the creation of the medical report. The modality is an imaging apparatus corresponding to the target inspection of the medical report. The data storage folder is information (storage location) indicating a folder in which data (such as a PDF file) of a medical report is stored. The creation date and time is a date and time when the medical report is created.

The first checking authority user ID is a user ID of a user who has the first checking authority for the associated medical report. The user having the first checking authority is, for example, a requesting doctor or a doctor belonging to a division (medical department) to which the requesting doctor belongs. That is, the first checking authority is an authority for a requesting doctor (second user) who has requested the creation of a medical report (interpretation report) or a doctor in a medical department to which the requesting doctor (second user) belongs to check the medical report (interpretation report).

The second checking authority user ID is a user ID of a user who has the second checking authority for the associated medical report. The user having the second checking authority is a user other than the user having the first checking authority. The user having the second checking authority is, for example, a nurse in a division to which the requesting doctor belongs, a doctor belonging to a division other than the division to which the requesting doctor belongs (a doctor in another medical department), or the like. That is, the second checking authority is an authority for the requesting doctor (second user) and a user other than the doctor in the medical department to which the requesting doctor (second user) belongs to check the medical report (interpretation report).

The first checking status flag is a flag indicating a checking status of the medical report by the user having the first checking authority. As the first checking status flag, 0: unchecked (unread) and 1: checked (read) are used. The default value of the first checking status flag is “0”.

The report check history table T7 is a table for managing the following. To be specific, whether or not the medical report registered in the report information table T6 is checked by the user having the first checking authority or the user having the second checking authority is managed. In the report check history table T7, a target report ID, a checking user ID, a report checking date and time, and an authority type at the time of checking are associated with each other.

The target report ID is a report ID of a target medical report. The checking user ID is a user ID of a user who has checked the medical report. The user who has checked the medical report is a user who has pressed a check button 326c which will be described later. The report checking date and time is date and time when the medical report has been checked. The authority type at the time of checking is the authority type (the first checking authority or the second checking authority) corresponding to the checking user when the medical report is checked.

The report reference history table T8 is a table for managing a history that the medical report registered in the report information table T6 has been referred to by a predetermined user. In the report reference history table T8, a target report ID, a reference user ID, and a report reference date and time are associated with each other.

The target report ID is a report ID of a target medical report. The reference user ID is a user ID of a user who has referred to the medical report. The user who has referred to the medical report is, for example, a user who has caused a medical report to be displayed. The report reference date and time is date and time when the medical report is referred to.

<1-2. Configuration of Client Terminal>

The client terminal 30 is a computer device such as a personal computer (PC) used by a doctor, a nurse, or the like A doctor, a nurse, or the like browses medical information such as a medical report on the client terminal 30.

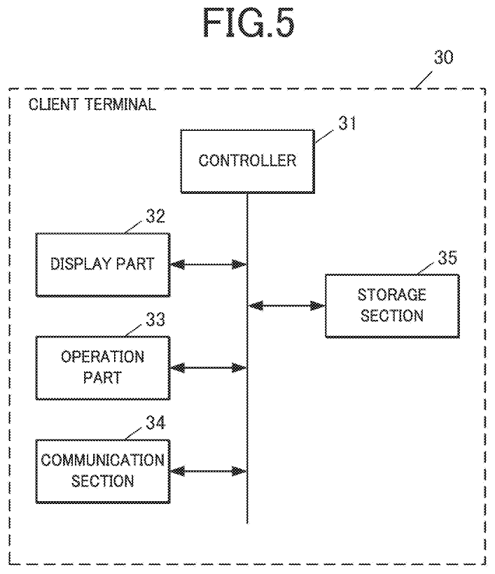

FIG. 5 shows the functional configuration of the client terminal 30.

As illustrated in FIG. 5, the client terminal 30 includes a controller 31, a display part 32, an operation part 33, a communication section 34, a storage section 35, and the like. Each unit of the client terminal 30 is connected by a bus.

The controller 31 includes a CPU, a ROM, a RAM, and the like, and integrally controls the processing operation of each unit of the client terminal 30. Specifically, the CPU reads various processing programs stored in the ROM, develops the programs in the RAM, and performs various processes in cooperation with the programs.

The display part 32 includes a monitor such as a liquid crystal display (LCD), and displays various screens in accordance with an instruction of a display signal input from the controller 31.

The operation part 33 includes a keyboard having cursor keys, character/numeral input keys, various function keys, and the like, and a pointing device such as a mouse. The operation part 33 outputs an operation signal input by a key operation on the keyboard or a mouse operation to the controller 31.

The communication section 34 includes a network interface and the like, and transmits and receives data to and from an external device connected via the communication network N.

The storage section 35 includes an HDD, a nonvolatile semiconductor memory, or the like, and stores various types of data.

<2. Operation of Medical Information Processing System>

Next, operations of the medical information processing system 100 will be described.

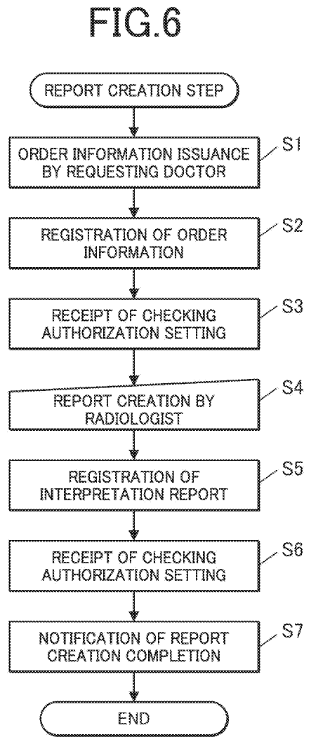

FIG. 6 is a flowchart illustrating a report creation step of creating a medical report which is executed in the medical information processing system 100. The medical report is assumed to be an interpretation report.

(Report Creation Step)

The electronic medical record server 10 transmits (issues) the order information of the interpretation report to the medical information processing apparatus 20 and the client terminals 30 used by the radiologist in response to the operation instruction from the client terminals 30 used by the requesting doctor (step S1). The order information includes, for example, the patient ID, the name of the requesting doctor, the requesting doctor user ID, the requesting division, the receipt number, and the modality.

Next, the controller 21 of the medical information processing apparatus 20 acquires the order information from the electronic medical record server 10 via the communication section 22. Next, the controller 21 registers the acquired order information in the report information table T6 (step S2).

To be specific, the controller 21 adds a new record to the report information table T6. Next, the controller 21 registers the patient ID, the name of the requesting doctor, the requesting doctor user ID, the requesting division, the receipt number, and the modality in association with the newly generated report ID.

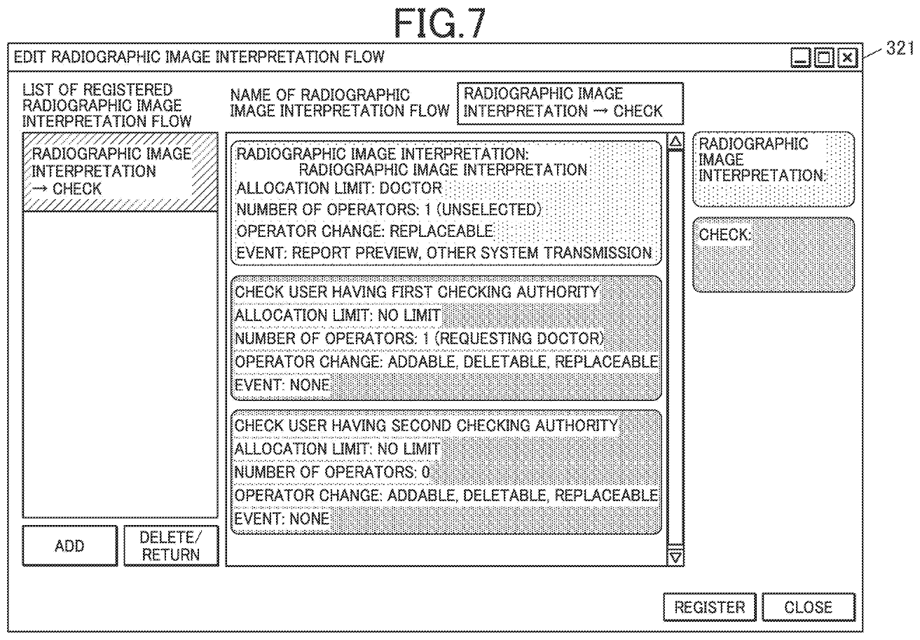

Next, the controller 21 allows the display part 32 of the client terminal 30 used by the requesting doctor to display an interpretation flow editing screen 321 illustrated in FIG. 7. Next, the controller 21 receives setting of checking authority in response to an operation instruction from the operation part 33 by the requesting doctor (step S3).

FIG. 7 illustrates an example of the image interpretation flow editing screen 321. FIG. 7 illustrates an example of initial setting in which it is preset to specify a requesting doctor as a user having the first checking authority and to specify no user having the second checking authority.

In the initial setting, no user having the first checking authority may be set to be specified.

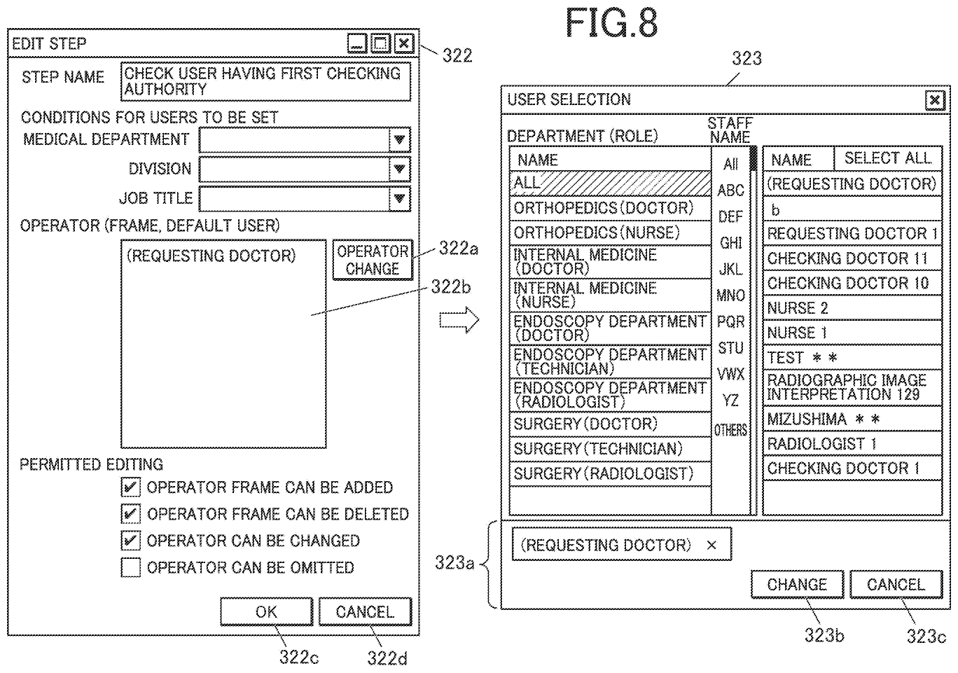

FIG. 8 illustrates an example of a case of changing the setting of the checking authority illustrated in FIG. 7.

In a case where the step editing is instructed from the operation part 33 on the interpretation flow editing screen 321, the controller 21 causes the display part 32 to display a step editing screen 322 illustrated in FIG. 8. Next, when an operator change button 322a in the step editing screen 322 is pressed, the controller 21 causes the display part 32 to display a user selection screen 323. Next, the controller 21 receives selection of the user having the first checking authority and/or the user having the second checking authority on the user selection screen 323. In the example illustrated in FIG. 8, the controller 21 receives the selection of the user having the first checking authority.

The controller 21 displays the user name of the currently selected user in a display area 323a of the user selection screen 323.

Next, if the cancel button 323c on the user selection screen 323 is pressed, the controller 21 returns to the step editing screen 322 without changing the setting of the checking authority.

On the other hand, when the change button 323b in the user selection screen 323 is pressed, the controller 21 displays the user name of the currently selected user in the operator field 322b of the step editing screen 322.

Next, when a cancel button 322d in the step editing screen 322 is pressed, the controller 21 does not store the setting change of the checking authority and closes the step editing screen 322. In other words, in this case, the setting of the checking authority is not changed.

On the other hand, if an OK button 322c in the step editing screen 322 is pressed, the controller 21 allows the image interpretation flow editing screen 321 to display the user name of the user selected in the user selection screen 323. Specifically, the user name of the selected user is displayed as the user having the first checking authority or the user having the second checking authority.

Next, the controller 21 performs the following processing based on the setting of the checking authority received on the interpretation flow editing screen 321. More specifically, the controller 21 performs processing for associating the first checking authority user ID and the second checking authority user ID with the record added in step S2.

More specifically, the controller 21 associates the user ID of the user selected as the user having the first checking authority with the record added in step S2 as the first checking authority user ID. The controller 21 associates the user ID selected as the user having the second checking authority with the record added in step S2 as the second checking authority user ID.

As described above, the controller 21 functions as a setting unit that can set the first checking authority and the second checking authority. The first checking authority is authority to check an interpretation report created by a radiologist (first user) who has interpreted a medical image of a subject. The second checking authority is a checking authority for an interpretation report different from the first checking authority.

In step S3, the setting of the user having the second checking authority may be omitted.

The controller 21 may notify the client terminal 30 used by the user having the first checking authority set in step S3 that the checking authority is set. Alternatively, the controller 21 may notify the client terminal 30 used by the user having the second checking authority set in step S3 that the checking authority is set.

That is, the controller 21 functions as a second controller when a user having the second checking authority is set. Specifically, the controller 21 realizes a second control step in which the mode of the notification to the user having the first checking authority is different from the mode of the notification to the user having the second checking authority.

The controller 21 may notify both of the following users that the checking authority is set. The users who may be notified of the setting specifically include a user who is set to have the first checking authority and a user who is set to have the second checking authority.

Next, the controller 31 of the client terminal 30 used by the radiologist acquires the order information from the electronic medical record server 10 via the communication section 34. The radiologist creates an interpretation report on the client terminal 30 based on the order information (step S4).

When the radiologist creates the interpretation report, the controller 21 acquires the interpretation report through the communication section 22. The interpretation report includes a report ID, a receipt number, and the like. Next, the controller 21 causes the storage section 24 to store the acquired interpretation report and registers information on the interpretation report in the report information table T6 (step S5).

To be specific, the controller 21 specifies a corresponding record from the report information table T6 based on the report ID, the receipt number, and the like of the interpretation report. Next, the controller 21 registers the data storage folder and the creation date and time in the specified record. Here, the controller 21 sets the first checking status flag to “0: Unchecked”.

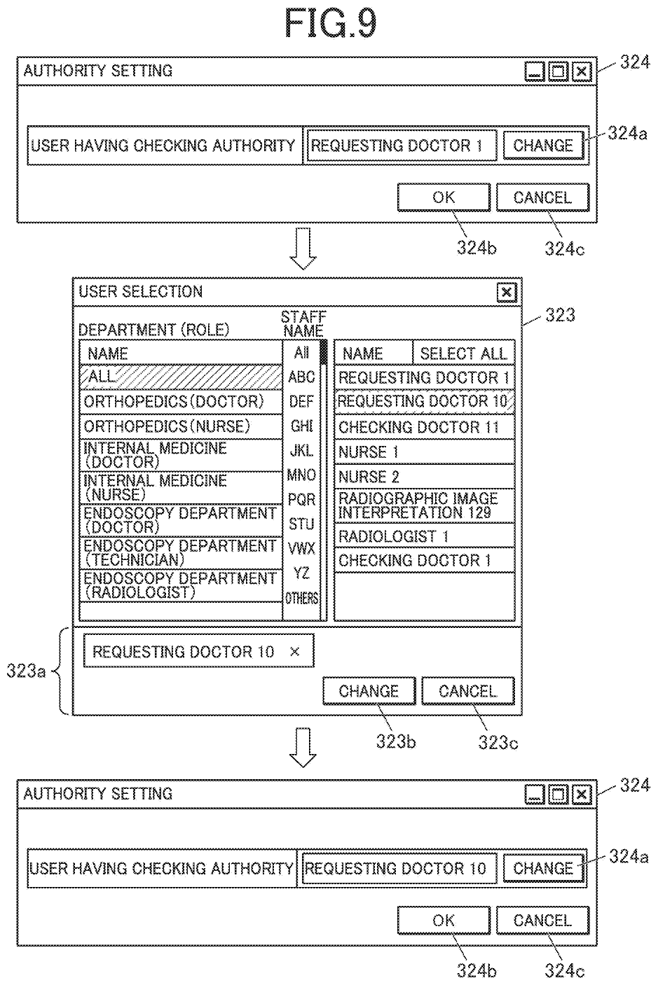

Next, the controller 21 allows the display part 32 of the client terminal 30 used by the radiologist to display an authority setting screen 324 illustrated in FIG. 9. Next, the controller 21 receives setting of checking authority in response to an operation instruction from the operation part 33 by the radiologist (step S6).

If the radiologist determines that the checking authority set in step S3 is not appropriate and/or is not sufficient the radiologist changes the setting of the checking authority.

If the setting of the checking authority received in step S3 is changed in step S6, the controller 21 changes the first checking authority user ID and/or the second checking authority user ID associated with the corresponding record based on the setting change of the checking authority.

FIG. 9 is an example showing a case where the user having the first checking authority is changed from “Requesting Doctor 1” to “Requesting Doctor 10”.

In FIG. 9, if a change button 324a in the authority setting screen 324 is pressed, the controller 21 allows the display part 32 to display a user selection screen 323 which is similar to that in FIG. 8. Next, the controller 21 receives selection of the user having the first checking authority and/or the user having the second checking authority on the user selection screen 323.

When a cancel button 323c in the user selection screen 323 is pressed, the controller 21 does not change the setting of the checking authority and returns the display of the display part 32 to the authority setting screen 324.

On the other hand, when a change button 323b in the user selection screen 323 is pressed, the controller 21 allows the username of the currently selected user to be displayed on the authority setting screen 324.

Next, if a cancel button 324c on the authority setting screen 324 is pressed, the controller 21 closes the authority setting screen 324 without saving the setting change of the checking authority. In other words, when the cancel button 324c on the authority setting screen 324 is pressed, the controller 21 closes the authority setting screen 324 without changing the setting of the checking authority.

On the other hand, when an OK button 324b in the authority setting screen 324 is pressed, the controller 21 changes the first checking authority user ID and/or the second checking authority user ID associated with the corresponding record to the user ID of the user selected in the user selection screen 323.

FIG. 9 illustrates an example in which the user selected on the user selection screen 323 is “Requesting Doctor 10”. When the user ID of “Requesting Doctor 10” matches the requesting doctor user ID registered in the corresponding record, or when the division to which “Requesting Doctor 10” belongs matches the requesting division registered in the corresponding record, the controller 21 sets “Requesting Doctor 10” as the user having the first checking authority. That is, the user who has the first checking authority is changed from “Requesting Doctor 1” to “Requesting Doctor 10”. Therefore, the controller 21 changes the first checking authority user ID registered in the corresponding record from the user ID of “Requesting Doctor 1” to the user ID of “Requesting Doctor 10”.

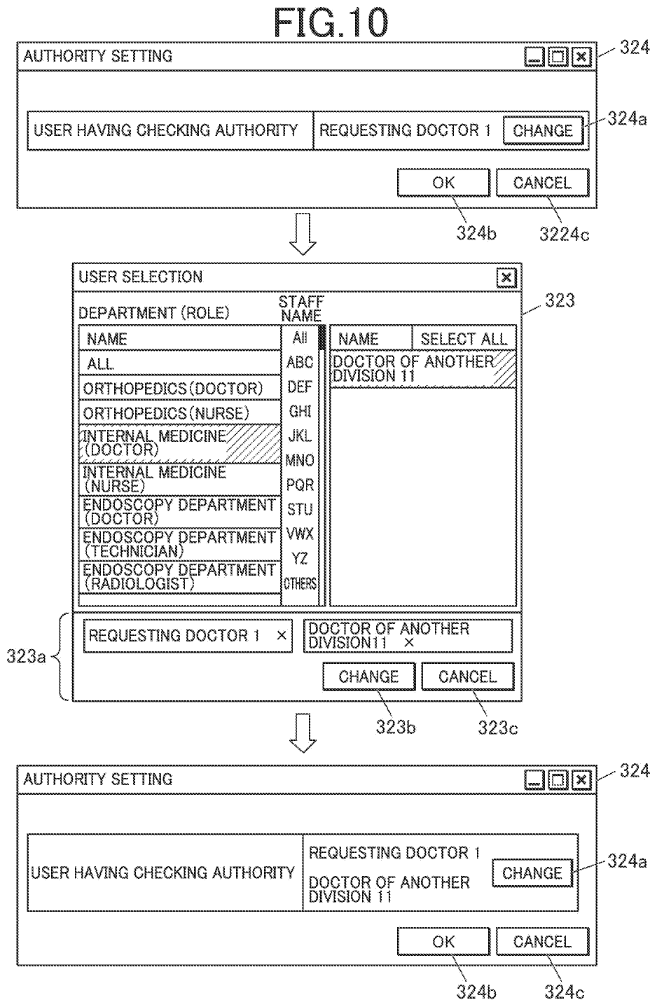

FIG. 10 shows an example in which “Doctor of Another Division 11” is added as the user having the second checking authority when “Requesting Doctor 1” is set as the user having the first checking authority.

In the example illustrated in FIG. 10, the users selected on the user selection screen 323 are “Requesting Doctor 1” and “Doctor of Another Division 11”. “Requesting Doctor 1” is set in advance as the user having the first checking authority. It is assumed that the division to which “Doctor of Another Division 11” belongs is one other than the requesting division registered in the corresponding record. In this case, the controller 21 sets “Doctor of Another Division 11” as the user having the second checking authority. That is, “Doctor of Another Division 11” is added as the user having the second checking authority. Therefore, the controller 21 registers the user ID of “Doctor of Another Division 11” as the second checking authority user ID in the corresponding record.

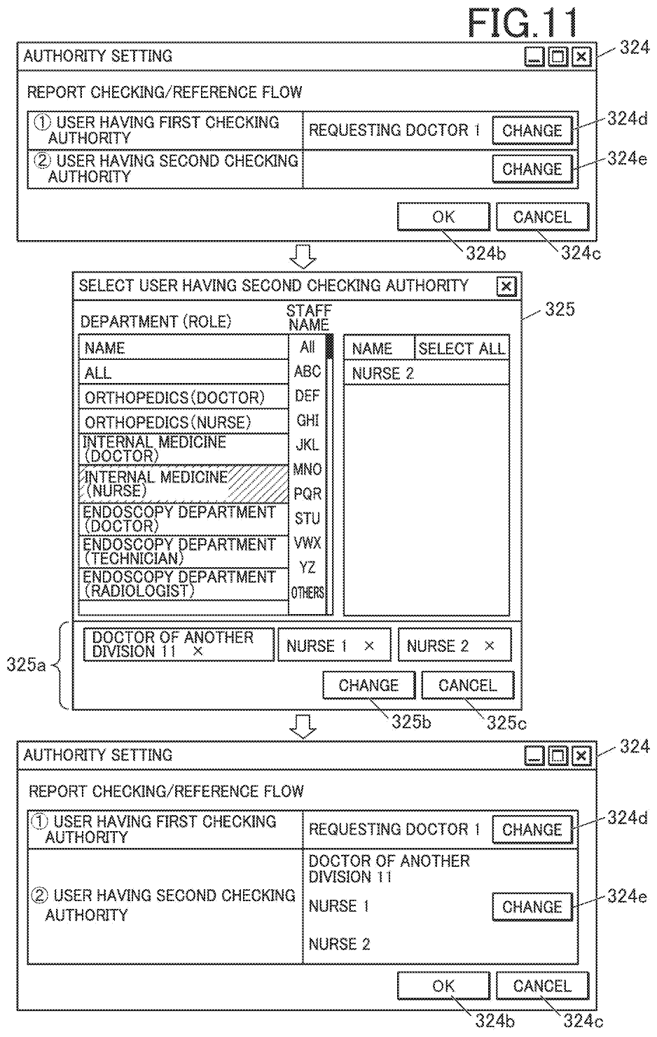

As shown in FIG. 11, the controller 21 may display the user having the first checking authority and the user having the second checking authority in an identifiable manner on the authority setting screen 324. As illustrated in FIG. 11, the controller 21 may set the order of checking the medical report on the authority setting screen 324 such that the user having the first authority is the first user, and the user having the second authority is the next user.

If a change button 324d in the authority setting screen 324 is pressed, the controller 21 causes the display part 32 to display a selection screen (not illustrated) of the user having the first authority. Next, the controller 21 receives the selection of the user having the first checking authority on the selection screen of the user having the first checking authority.

When a change button 324e in the authority setting screen 324 is pressed, the controller 21 causes the display part 32 to display the selection screen 325 of the user having the second checking authority. Next, the controller 21 receives the selection of the user having the second checking authority on the selection screen 325 of the user having the second checking authority.

FIG. 11 shows an example in which, when “Requesting Doctor 1” is set as the user having the first checking authority. “Doctor of Another Division 11”. “Nurse 1”, and “Nurse 2” are added as the users having the second checking authority.

The controller 21 displays the user name of the currently selected user in the display area 325a of the selection screen 325 of the user having the second checking authority.

Next, if a cancel button 325c in the selection screen 325 of the user having the second checking authority is pressed, the controller 21 does not change the setting of the checking authority and changes the display of the display part 32 back to the authority setting screen 324.

On the other hand, if a change button 325b in the selection screen 325 of the user having the second checking authority is pressed, the controller 21 displays the user name of the currently selected user in the field of the user having the second checking authority of the authority setting screen 324.

In the example illustrated in FIG. 11, the users selected on the selection screen 325 of a user having the second authority are “Doctor of Another Division 11”, “Nurse 1”, and “Nurse 2”. In this case, the controller 21 sets “Doctor of Another Division 11”, “Nurse 1”, and “Nurse 2” as the users having the second checking authority. That is. “Doctor of Another Division 11”, “Nurse 1”, and “Nurse 2” are added as users having the second checking authority. Therefore, the controller 21 registers the user IDs of “Doctor of Another Division 11”, “Nurse 1”, and “Nurse 2” in the corresponding record as the second checking authority user ID.

In step S6, the controller 21 may notify the client terminal 30 used by the newly set user having the first checking authority that the checking authority is set. Alternatively, in step S6, the controller 21 may notify the client terminal 30 used by the newly set user having the second checking authority that the checking authority is set.

The controller 21 may notify both of the following users that the checking authority is set. The users who may be notified of the setting specifically include a newly set user having the first checking authority and a newly set user having the second checking authority.

Next, the controller 21 notifies the electronic medical record server 10 and the client terminal 30 used by the user set as having the first checking authority or the second checking authority at the time of step S6 that the interpretation report has been created (step S7). Next, the controller 21 ends the present processing.

That is, in a case where a user having the second checking authority is set, the controller 21 makes the mode of the notification to the user having the first checking authority and the mode of the notification to the user having the second checking authority different from each other.

In step S7, when notifying the electronic medical record server 10 that the interpretation report has been created, the controller 21 may perform the following processing. To be specific, the step S7 may include processing of notifying the electronic medical record server 10 of the first checking authority user ID and/or the second checking authority user ID corresponding to the report ID of the interpretation report.

In step S7, the controller 21 may notify both the following users that the interpretation report has been created. The users who may be notified specifically include a user who is set to have the first checking authority and a user who is set to have the second checking authority at the time of the step S6.

Next, report checking status management processing executed in the medical information processing apparatus 20 will be described.

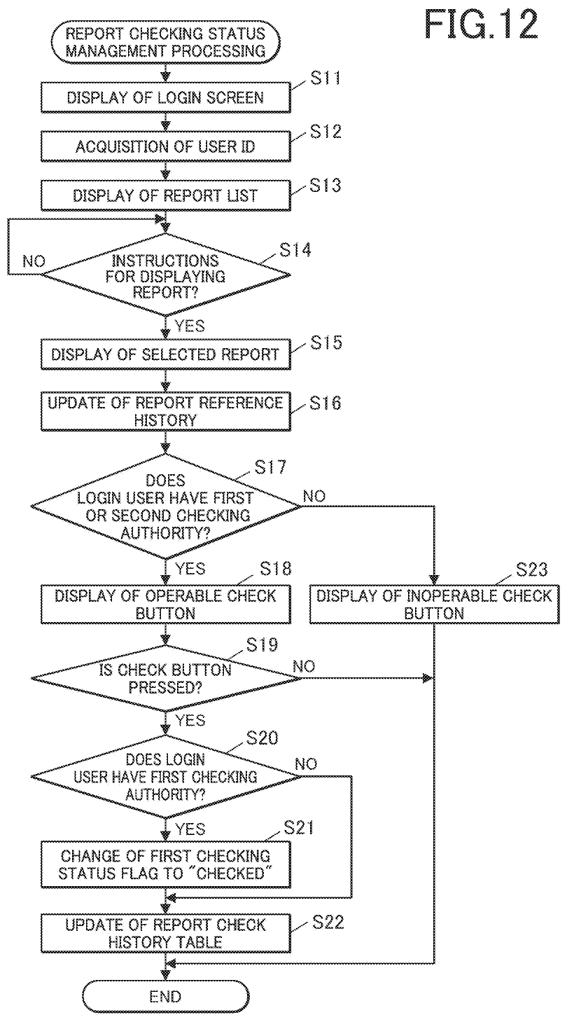

FIG. 12 is a flowchart illustrating the report checking status management processing. The CPU of the controller 21 realizes this processing by software processing in cooperation with a control program stored in the ROM.

### (Report Checking Status Management Processing)

The operator accesses the medical information processing apparatus 20 via the operation part 33 of the client terminal 30. In response to the access operation, the controller 21 of the medical information processing apparatus 20 transmits display information for displaying a login screen to the client apparatus 30 via the communication section 22 (step S11).

A login screen is displayed on the display part 32 of the client terminal 30. The login screen includes a user ID input field.

In the client terminal 30, the operator inputs the user ID of the operator himself/herself (login user) via the operation part 33. Tien, the controller 21 of the medical information processing apparatus 20 acquires the input user ID via the communication section 22 (step S12).

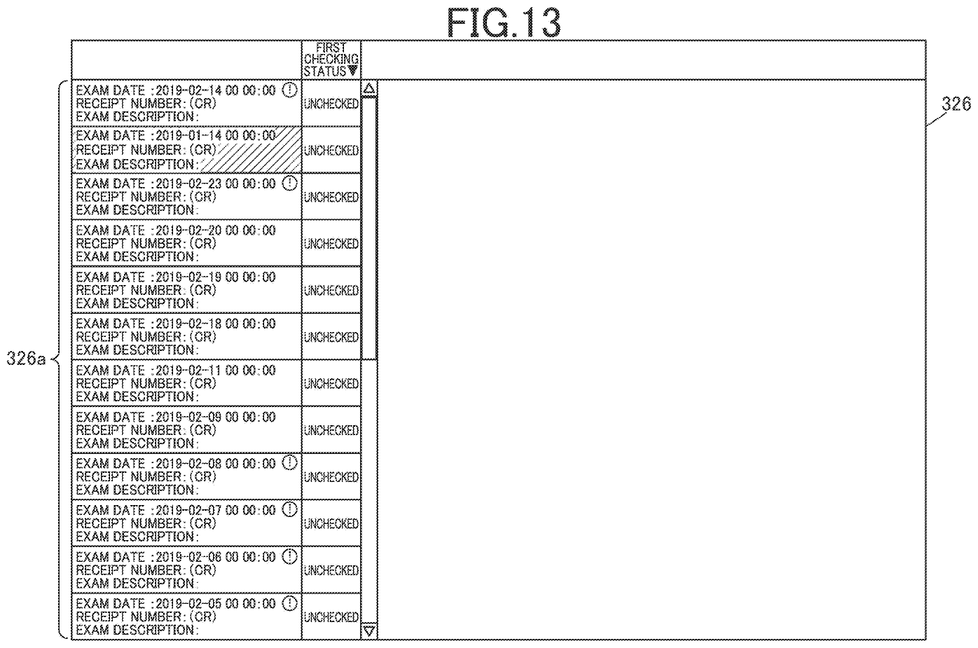

Next, the controller 21 of the medical information processing apparatus 20 causes the display part 32 of the client terminal 30 to display a report display screen 326 shown in FIG. 13. Next, the controller 21 allows a list of medical reports stored in the storage section 24 to be displayed in the display area 326a (step S13).

In the example illustrated in FIG. 13, the report in which the item of the first checking status is “unchecked” indicates that the “first checking status flag” associated with the “report ID” is “0: Unchecked”.

Next, the controller 21 determines the following (step S14). Specifically, it is determined whether or not an instruction to display any report of the list of medical reports displayed in step S13 has been given by an operator's operation on the operation part 33.

If the display instruction of any report is not performed (step S14; NO), the controller 21 repeats the processing of step S14.

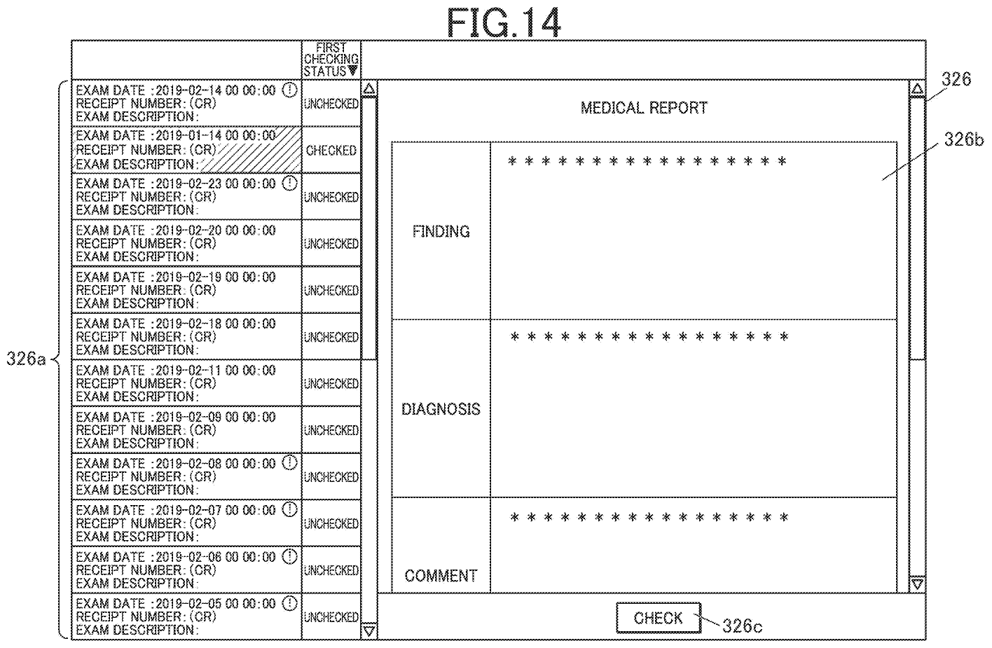

On the other hand, if an instruction to display one of the reports has been given (step S14; YES), the controller 21 allows the selected report to be displayed in the display area 326b of the report display screen 326 as illustrated in FIG. 14 (step S15).

When the controller 21 can receive specification of a report ID by an operation performed on the operation part 33 by the operator, steps S13 and S14 may be omitted.

Next, the controller 21 updates the report reference history table T8 in the storage section 24 (step S16).

To be specific, the controller 21 adds a new record to the report reference history table T8. The controller 21 stores the “report ID” of the selected report in the “target report ID” field of the new record. The controller 21 stores the “user ID” of the login user in the “reference user ID” field of the new record. The controller 21 stores the current date and time acquired from the clock 23 in the “report reference date and time” field of the new record.

Next, the controller 21 determines whether or not the login user is a user having the first checking authority or a user having the second checking authority (step S17). To be specific, the controller 21 refers to the report information table T6 in the storage section 24. Next, the controller 21 determines whether or not the “user ID” of the login user matches the first checking authority user ID or the second checking authority user ID associated with the “report ID” of the selected report.

If the login user has the first checking authority or the second checking authority (step S17; YES), the controller 21 displays a check button 326c so as to be operable (pressable), as illustrated in FIG. 14 (step S18). The term “pressable” means that the status can be changed to a pressed status by an operation from the operation part 33.

As shown in FIG. 11, when the order of checking the medical report is set, the controller 21 performs the following processing in step S18. To be specific, until the check button 326c is pressed by the user having the first checking authority, the controller 21 causes the check button 326c to be displayed so as not to be pressable by the user having the second checking authority.

Next, the controller 21 determines whether or not the check button 326c displayed in step S18 has been pressed by an operation from the operation part 33 (step S19).

If the check button 326c is pressed (step S19; YES), the controller 21 determines whether or not the login user is a user having the first checking authority (step S20). That is, the controller 21 determines the checking authority possessed by the user. The controller 21 functions as a determiner that realizes a determination step.

If the login user is a user having the first checking authority (step S20; YES), the controller 21 changes the “first checking status flag” associated with the “report IDs” of the selected reports to “1: Checked” in the report information table T6 of the storage section 24 (step S21).

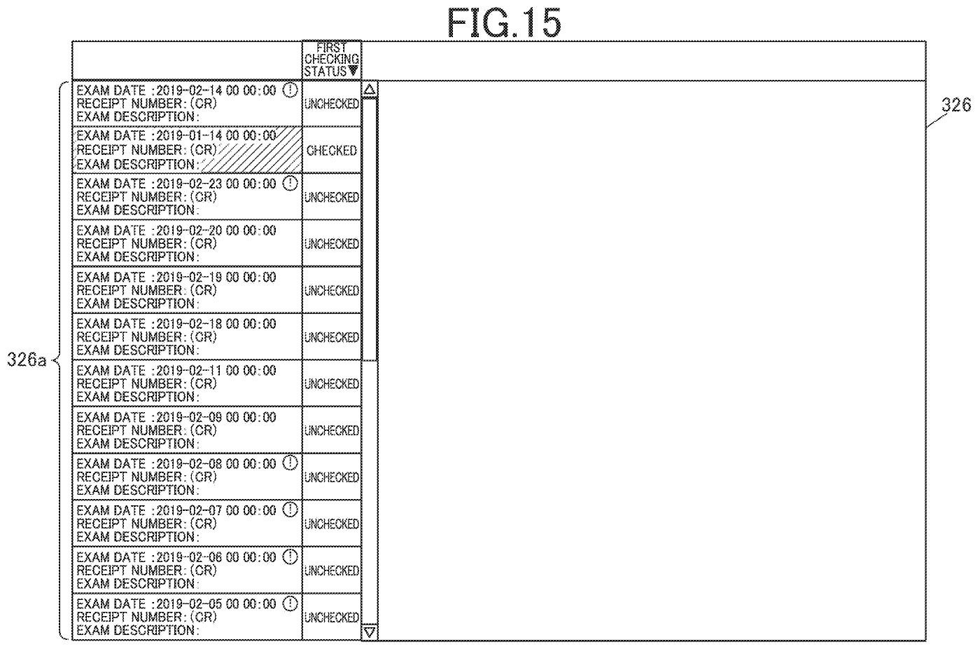

Next, as illustrated in FIG. 15, the controller 21 changes “unchecked” of the item of the first checking status of the selected report to “checked” in the list of medical reports of the report display screen 326.

On the other hand, if the login user is not a user having the first checking authority (step S20; NO), that is, if the login user is a user having the second checking authority, the controller 21 moves the processing to Step S22.

Next, the controller 21 updates the report check history table T7 in the storage section 24 (step S22) and ends the processing.

To be specific, the controller 21 stores, in the report check history table T7, “target report ID”, “checking user ID”, “report checking date and time”, and “authority type at the time of checking” in an associated manner. The controller 21 stores the “report ID” of the selected report in the “target report ID” field. The controller 21 stores the “user ID” of the login user in the “checking user ID” field. The controller 21 stores the current date and time acquired from the clock 23 in the “report checking date and time” field. The controller 21 stores the “first checking authority” or “second checking authority”, which is the checking authority of the login user, in the “authority type at the time of checking” field.

When the login user does not have the first checking authority and has the second checking authority, the controller 21 functions as a first controller. To be specific, the controller 21 updates the report check history table T7 without changing the “first checking status flag”, thereby realizing a first control step of enabling the check by the user having the second checking authority.

As described above, in a case where the check button 326c is pressed by the login user having the first checking authority, the controller 21 changes the “first checking status flag” to “1: checked”. Next, in this case, the controller 21 stores the “first checking authority” in the “authority type at the time of checking” field of the report check history table T7. Next, the controller 21 changes the item of the first checking status of the corresponding report from “unchecked” to “checked” in the list of the medical reports on the report display screen 326.

On the other hand, when the check button 326c is pressed by the login user having the second checking authority, the controller 21 does not change the “first checking status flag”. Next, in this case, the controller 21 stores the “second checking authority” in the “authority type at the time of checking” field of the report check history table T7.

That is, the controller 21 distinguishably outputs the display of the check history in a case where the user having the first checking authority performs the checking and the display of the check history in a case where the user having the second checking authority performs the checking. The controller 21 functions as an output section that implements an output step.

Outputting, in a distinguishable manner, the display of the check history in a case where the user having the first checking authority performs checking and the display of the check history in a case where the user having the second checking authority performs checking may be displaying as follows.

For example, the controller 21 may display the list of the medical reports on the report display screen 326 such that the thickness, color, transparency, and the like of the characters am different between the report checked by the user having the first checking authority and the report checked by the user having the second checking authority. Alternatively, in the list of medical reports on the report display screen 326, the controller 21 may assign a symbol or a character to a report that has been checked by the user having the first checking authority or a report that has been checked by the user having the second checking authority. Alternatively, in the list of medical reports on the report display screen 326, the controller 21 may hide a report that has been checked by the user having the first checking authority or a report that has been checked by the user having the second checking authority.

If the login user is not the user having the first checking authority or the user having the second checking authority (step S17; NO), the controller 21 displays the check button 326c so as no to be pressable (step S23) and ends the present processing. The term “not pressable” means that the status cannot be changed to a pressed status by an operation from the operation part 33. For example, a button can be grayed out to indicate that it is not an operation target. Instead of displaying the check button 326c so as not to be pressable, the check button 326c may be hidden.

If the check button 326c is not pressed (step S19; NO), the controller 21 ends the present processing.

Next, a case where the checking authority of the report in which the “first checking status flag” is “checked” is set will be described. A report in which the “first checking status flag” is “checked” is a report checked by a user having the first checking authority.

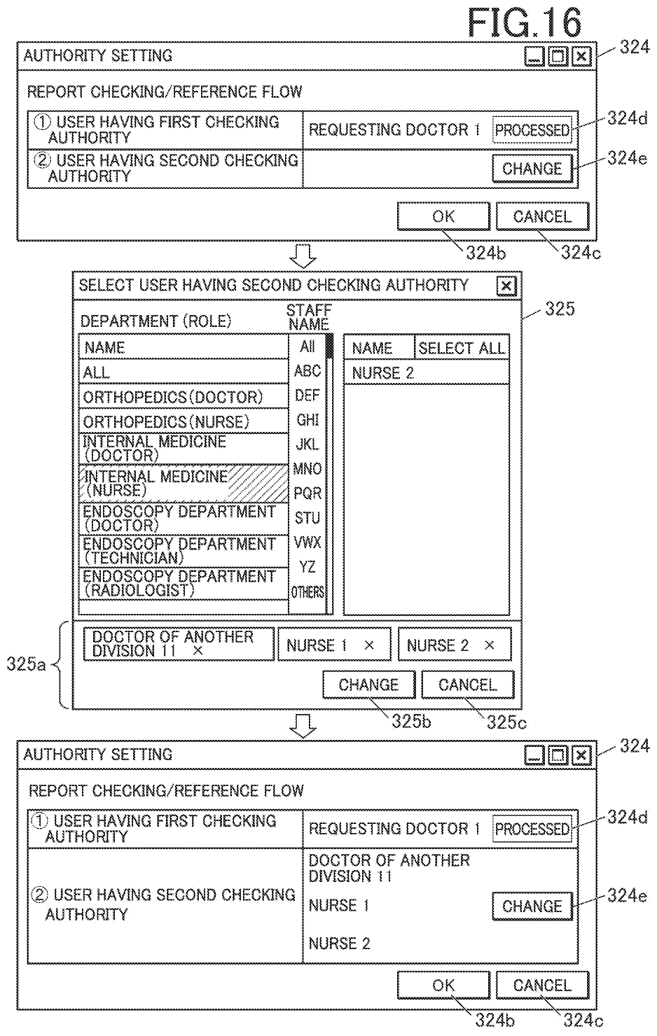

For example, it is assumed that the following instruction is given from the operation part 33 of the client terminal 30 used by the user having the first checking authority. Specifically, the instruction from the operation part 33 is an instruction to receive the setting of the checking authority of the report checked by the user having the first checking authority. In this case, the controller 21 allows the display part 32 to display an authority setting screen 324 illustrated in FIG. 16. Next, the controller 21 receives setting of a checking authority in response to an operation instruction from the operation part 33.

For example, when the user having the first checking authority determines that the checking authority set in the report is not appropriate and/or is not sufficient, the user changes the setting of the checking authority.

As illustrated in FIG. 16, the controller 21 causes a change button 324d for changing the user having the first checking authority to be displayed so as not to be pressable in the report checked by the “Requesting Doctor 1”. The “Requesting Doctor 1” is a user who has the first checking authority. Instead of displaying the change button 324d so as not to be pressable, the controller 21 may hide the change button 324d. As illustrated in FIG. 16, the controller 21 may cause the change button 324d to display letters “processed”.

That is, the controller 21 performs control such that the user having the first checking authority cannot be changed in the report checked by “Requesting Doctor 1”.

When the user having the first checking authority presses the change button 324e in the authority setting screen 324 by using the operation part 33, the controller 21 receives the selection of the user having the second checking authority as in the case of FIG. 11.

In FIG. 16, similarly to the case of FIG. 11, “Doctor of Another Division 11”, “Nurse 1”, and “Nurse 2” are added as the users laving the second checking authority. In this case, the controller 21 registers the user IDs of “Doctor of Another Division 11”, “Nurse 1”, and “Nurse 2” as the second checking authority user ID in the record of the report.

The controller 21 may perform control such that a user other than the “Requesting Doctor 1” can be additionally set as a user having the first checking authority for a report checked by the “Requesting Doctor 1”.

Similarly to step S6 of the report creation step, the controller 21 may notify the client terminal 30 used by the newly set user having the first checking authority that the checking authority is set. Alternatively, similarly to step S6 in the report creation step, the controller 21 may notify the client terminal 30 used by the newly set user having the second checking authority that the checking authority is set.

The controller 21 may notify both of the following users that the checking authority is set. The users who may be notified of the settings include, specifically, a newly set user having the first checking authority and a newly set user having the second checking authority.

In the report checked by the user having the second checking authority, the controller 21 may perform control such that the user having the second checking authority who has performed the check cannot be changed. The report checked by the user having the second checking authority is a report in which the “second checking authority” is stored in the “authority type at the time of checking” field of the report check history table T7.

In a case where the interpretation report is checked by the user having the first checking authority or the second checking authority, the controller 21 functions as a third controller. Specifically, the controller 21 realizes a third control step of prohibiting the change (deletion) of the first checking authority or the second checking authority for the user who has performed the check.

As described above, in the report checked by the user having the first checking authority or the user having the second checking authority, when the checking authority is set, the controller 21 does not update the report check history table T7.

When the first checking authority or the second checking authority of the user who has not checked the interpretation report is changed after a user having the first checking authority or the second checking authority has checked the interpretation report, the controller 21 functions as a fourth controller. To be specific, the controller 21 realizes a fourth control step of prohibiting the deletion of the information (check history) stored in the report check history table T7 when the user having the first checking authority or the second checking authority has checked the interpretation report.

The checking authority may be set before step S20 of the report checking status management processing. That is, the checking authority may be set in a status where the report has been displayed on the report display screen 326 and has not been checked by the user having the first checking authority or the user having the second checking authority.

That is, the setting process of setting the checking authority may be performed at the time of request to create a report (step S3 of the report creating process). Alternatively, the setting step may be performed when a report is created (report creation step S1). Alternatively, the setting step may be performed in a status where the report has been displayed on the report display screen 326 and has not been checked by the user having the first checking authority or the user having the second checking authority. Alternatively, the setting step may be performed after check by the user having the first checking authority or the user having the second checking authority.

A case where the checking authority is set after the completion of the report creation step will be described. In this case, the controller 21 may notify the client terminal 30 used by the newly set user having the first checking authority that the medical report has been created. Alternatively, the controller 21 may notify the client terminal 30 used by the newly set user having the second checking authority that the medical report has been created. Furthermore, in this case, the controller 21 may notify the electronic medical record server 10 of the newly set first checking authority user ID and/or second checking authority user ID.

<3. Effects>

As described above, according to the present embodiment, the medical information processing apparatus 20 includes the setting unit (controller 21) capable of setting the first checking authority which is the authority to check the interpretation report created by the first user (radiologist) who has interpreted the medical image of the subject and the second checking authority which is the authority to check the interpretation report and different from the first checking authority.

Therefore, it is possible to set a plurality of types of the checking authority of the interpretation report, and thus it is possible to check the interpretation report in accordance with teds.

The medical information processing apparatus 20 includes an output section (controller 21) that output display of a check history in a case where a user having a first checking authority performs check and display of a check history in a case where a user having a second checking authority performs check in an identifiable manner.

Therefore, the check by the user having the first checking authority and the check by the user having the second checking authority can be distinguished from each other and can be managed in accordance with needs.

The medical information processing apparatus 20 includes a determiner (the controller 21) that determines the checking authority possessed by the user. The medical information processing apparatus 20 includes the first controller (the controller 21) that enables the user having the second checking authority to performing check when the user does not have the first checking authority but has the second checking authority.

Therefore, a user who does not have the first checking authority can also check the interpretation report.

The medical information processing apparatus 20 includes the second controller (the controller 21) that makes the notification to the user having the first checking authority and the notification to the user having the second checking authority different from each other when the user having the second checking authority is set.

Therefore, the user having the second checking authority can also recognize that the checking authority has been set or the report has been created.

In the medical information processing apparatus 20, the first checking authority is an authority for a second user (requesting doctor) who has requested creation of an interpretation report or a doctor in a medical department to which the second user belongs to check the interpretation report. The second checking authority is an authority for user other than the second user and other than the doctor in the medical department to which the second user belongs to check the interpretation report.

Therefore, a user other than the requesting doctor and other than the doctor belonging to the group to which the requesting doctor belongs can also check the interpretation report.

The medical information processing apparatus 20 includes a third contoller (controller 21) that prohibits, in a case where the interpretation report is checked by a user having the first checking authority or the second checking authority, the deletion of the first checking authority or the second checking authority of the user who has performed the checking.

Therefore, it is possible to manage the check by the user having the first checking authority and the check by the user having the second checking authority in accordance with needs.

The medical information processing apparatus 20 includes a fourth controller (controller 21) that prohibits the deletion of the check history of the interpretation report by the user having the first checking authority or the second checking authority in a case where the first checking authority or the second checking authority of the user is changed after the interpretation report is checked by the user having the first checking authority or the second checking authority.

Therefore, it is possible to manage the check by the user having the first checking authority and the check by the user having the second checking authority in accordance with needs.

The description in the above embodiment is an example of the medical information processing system according to the present invention, and the present invention is not limited to this. The detailed configuration and detailed operation of each device constituting the system can also be appropriately changed without departing from the scope of the present invention.

For example, although the description has been given centering on the check of the medical report in the above embodiment, medical information other than the medical report may be the target to be checked.

In the above embodiment, the case where the control program of the present invention is a program executed by the medical information processing apparatus 20 in the medical information processing system 100 has been described. However, this program may be executed by another apparatus.

In the above-described embodiment, the user having the first checking authority intentionally performs an operation to set the medical information (medical report) to a checked status. The checked status is a status in which the “first checking status flag” is “checked”. However, the status may be automatically shifted to the checked status. For example, the controller 21 may detect that a medical report has been browsed and displayed (referred to) by a user having the first checking authority and automatically set the medical report as checked. Alternatively, in a case where the viewing time of the medical report by the user having the first checking authority is equal to or longer than a predetermined time, the medical report may be automatically set as checked.

Although the medical information processing system 100 includes the electronic medical record server 10 in the above embodiment, the medical information processing system 100 is not limited to this embodiment. The medical information processing system 100 may not include the electronic medical record server 10 and may include the medical information processing apparatus 20 that stores a created medical report in the storage section 24.

Although the example in which a ROM is used as a computer-readable medium storing a program for executing each process has been disclosed in the above description, the medium is not limited to this example. As the computer-readable medium, a nonvolatile memory such as a flash memory or a portable recording medium such as a CD-ROM can also be applied. Furthermore, a carrier wave may be applied as a medium for providing data of the program via a communication line.

Although embodiments of the present invention have been described and illustrated in detail, the disclosed embodiments am made for purposes of illustration and example only and not limitation. The scope of the present invention should be interpreted by terms of the appended claims.

1. A medical information processing apparatus comprising:
a hardware processor that sets first checking authority and second checking authority, the first checking authority being authority to check an interpretation report created by a first user who has interpreted a medical image of a subject, the second checking authority being authority to check the interpretation report and being different from the first checking authority.

2. The medical information processing apparatus according to claim 1,
wherein the hardware processor outputs a first display of a check history by a user having the first checking authority and a second display of a check history by a user having the second checking authority in a distinguishable manner.

3. The medical information processing apparatus according to claim 1,
wherein the hardware processor determines checking authority of a user, and
wherein, upon the user does not have the first checking authority and has the second checking authority, the hardware processor allows a user having the second checking authority to check the interpretation report.

4. The medical information processing apparatus according to claim 1,
wherein, upon a user having the second checking authority being set, the hardware processor makes a first mode of notification to a user having the first checking authority different from a second mode of notification to the user having the second checking authority.

5. The medical information processing apparatus according to claim 1,
wherein the first checking authority is authority for a second user who has requested creation of the interpretation report or a doctor of a medical department to which the second user belongs to check the interpretation report, and
wherein the second checking authority is authority for a user other than the second user and other than a doctor of a medical department to which the second user belongs to check the interpretation report.

6. The medical information processing apparatus according to claim 1,
wherein, upon a user having the first checking authority or the second checking authority checking the interpretation report, the hardware processor prohibits deletion of the first checking authority or the second checking authority of the user.

7. The medical information processing apparatus according to claim 1,
wherein, upon change of the first checking authority or the second checking authority of a user having the first checking authority or the second checking authority and having checked the interpretation report, the hardware processor prohibits deletion of a check history of the interpretation report by the user.

8. A medical information processing system comprising:
a hardware processor that sets a first checking authority and a second checking authority, the first checking authority being authority to check an interpretation report created by a first user who has interpreted a medical image of a subject, the second checking authority being authority to check the interpretation report and being different from the first checking authority.

9. A medical information processing method comprising:
setting a first checking authority and a second checking authority, the first checking authority being an authority to check an interpretation report created by a first user who has interpreted a medical image of a subject, the second checking authority being an authority to check the interpretation report and being different from the first checking authority.

10. A non-transitory computer-readable recording medium storing a program that causes a hardware processor of a computer to set a first checking authority and a second checking authority, the first checking authority being an authority to check an interpretation report created by a first user who has interpreted a medical image of a subject, the second checking authority being an authority to check the interpretation report and being different from the first checking authority.

11. The recording medium according to claim 10,
wherein the program causes the hardware processor to output a first display of a check history by a user having the first checking authority and a second display of a check history by a user having the second checking authority in a distinguishable manner.

12. The recording medium according to claim 10,
wherein the program causes the hardware processor to determine checking authority of a user, and
wherein, upon the user does not have the first checking authority and has the second checking authority, the program causes the hardware processor to allow a user having the second checking authority to check the interpretation report.

13. The recording medium according to claim 10,
wherein upon a user having the second checking authority being set, the program causes the hardware processor to make a first mode of notification to a user having the first checking authority different from a second node of notification to the user having the second checking authority.

14. The recording medium according to claim 10,
wherein the first checking authority is authority for a second user who has requested creation of the interpretation report or a doctor of a medical department to which the second user belongs to check the interpretation report, and
wherein the second checking authority is authority for a user other than the second user and other than a doctor of a medical department to which the second user belongs to check the interpretation report.

15. The recording medium according to claim 10, wherein, upon a user having the first checking authority or the second checking authority checking the interpretation report, the program causes the hardware processor to prohibit deletion of the first checking authority or the second checking authority of the user.

16. The recording medium according to claim 10, wherein, upon change of the first checking authority or the second checking authority of a user having the first checking authority or the second checking authority and having checked the interpretation report, the program causes the hardware processor to prohibit deletion of a check history of the interpretation report by the user.
